(19) United States
(12) Patent Application Publication
(10) Pub. No.: US 2023/0381506 A1
(43) Pub. Date: Nov. 30, 2023

(54) DEVICE FOR TREATING LONG-TERM OR CHRONIC PAIN THROUGH VAGUS NERVE STIMULATION AND USE THEREOF

(71) Applicant: Euronlabs AS, Strømmen (NO)

(72) Inventor: Kenneth Sanders, Kløfta (NO)

(21) Appl. No.: 18/032,976

(22) PCT Filed: Oct. 25, 2021
(86) PCT No.: PCT/NO2021/050221

§ 371 Date: Apr. 20, 2023

(30) Foreign Application Priority Data
Oct. 23, 2020 (NO) 20201159

(51) Int. Cl. A61N 1/36; A61B 5/0205; G16H 10/60; A61N 1/04

(52) U.S. Cl. CPC A61N 1/36021; A61N 1/36034; A61B 5/0205; G16H 10/60; A61N 1/0456

(57) ABSTRACT

It has been found that affecting the vagus nerve in mammals and humans with electrical impulses reduces and alleviates widespread chronic primary pain. For this end it has been developed a device for alleviating or removing pain sensation in an individual, the device comprising a source for electricity (I) for creating pulses with a width range of 5-1000 μs, frequency of 1-50 000 Hz, voltage within an interval of 1.0-50,000 V, and a current within an interval of 0.01-10 mA to at least two electrodes (3.3′, 4) for creating a voltage between said electrodes, said electrodes being placed on a location on the body of the individual for stimulating the vagus nerve, the at least two electrodes (3.3′4) creating a voltage difference between said electrodes when connected to the power source (I). Further, the device comprises sensors for monitoring and recording health measurements in order to optimize daily health, wherein the sensors are integrated in the device with electrodes that send data signals to a software application that collects and records health measurements.

### FIELD OF THE INVENTION

The present invention concerns a device for treating long-term or chronic pain and inflammation or other ailments through transcutaneous electrical stimulation of the vagus nerve in the mammalian body, preferably a human or simian organism.

### PRIOR ART

Stimulation of the vagus nerve has been known to provoke physical effects. As long back as practicing acupuncture therapies with needles placed at different locations of the body for alleviating diseases, such treatment has been known to have an effect on the body, all from treating mental afflictions to treating bodily conditions and afflictions. Such afflictions could be nausea, schizophrenia and other mental afflictions as well as more physical ailments such as stomach or bowel diseases (constipation/diarrhea), high or low blood pressure, etc. This has been described in scientific articles such as “Anti-inflammatory properties of the vagus nerve: potential therapeutic implications of vagus nerve stimulation” (J. Physiol. 594.20 (2016), pp. 5781-5790); “Vagus Nerve Stimulation for Treatment of Inflammation: Systematic Review of Animal Models and Clinical Studies”, Kwan et. al, Bioelectron. Med. 3: 1-6 (2016); “Vagus nerve stimulation: a new promising therapeutic tool in inflammatory bowel disease”, B. Bonaz et. al, 2017 The Association for the Publication of the Journal of Internal Medicine; “Vagus nerve stimulation improves working memory performance” L. Sun et. al; J. of Clin. and Exp. Neurophys; February 2017; “Enhanced recognition memory following vagus nerve stimulation in human subjects” K. B. Clark et. al, Nature Neurosci. Vol. 2, no. 1, January 1999; “Vagus nerve stimulation: from epilepsy to the cholinergic anti-inflammatory pathway”, B. Bonaz et. al, Neurogastroenterol. Motil. (2013) 25, 208-221; “Vagal stimulation Modulates Inflammation through a Ghrelin Mediated Mechanism in Traumatic Brain Injury”, V. Bansal et. al, Inflammation 2012, February 214-220; “Kilohertz frequency nerve block enhances anti-inflammatory effects of vagus nerve stimulation”, Y. A. Patel et. al, Scientific Reports 7: 39810/DOI: 10.1038/srep39810; “Specific vagus nerve stimulation parameters alter serum cytokine in the absence of inflammation”, T. Tsaava et. al, Bioelectronic Medicine (2020) 6:8; “Advances in the Treatment of Cholinergic Anti-Inflammatory Pathways in Gastrointestinal Diseases by Electrical Stimulation of Vagus Nerve” W. Lei et. al, Digestion, DOI: 10.1159/000504474; “Is there a role for vagus nerve stimulation in the treatment of posttraumatic stress disorder?”, Bioelectron. Med. (2018), 1(2), 95-99; “Transcutaneous Vagus Nerve Stimulation: A Promising Method for Treatment of Autism Spectrum Disorders”, Y. Jin et. al, Frontiers in Neuroscience, January 2017, Vol 10, Art. 609.

Some attempts have been made previously for presenting devices for stimulating the vagus nerve. There exists on the market a device called ActiPatch acting on the insole of the foot for affecting the vagus nerve. This device is, however, not suitable for affecting the vagus nerve through the ear, and this location also suggests that the foot is the more preferred limb to target. Also this device is presented as suitable for allay and recovery and not against chronic pain.

### INTRODUCTION/BACKGROUND FOR THE INVENTION

Long-term pain conditions are common, disabling for patients, and time-consuming for the health care services. Long term pain is, in the present context, considered to be pain experienced by the relevant patient/person/individual lasting over a period of at least 24 hours and such pain sensations are considered to be nerve stimulation of nerve receptors sending nerve signals of more or less discomfort or experienced unnatural situations to the brain. Pain is a subjective sensation normally causing the inflicted or influenced individual to take measures or acts to avoid or reduce said sensation. Pain may even be debilitating and inflict severe damage to the life quality of the inflicted or affected individual. Some of the effects of long term pain on the afflicted organism are low heart rate variability of the autonomic nervous system (Barakat, Ansam, N. Vogelzangs, Carmilla M M Licht, Rinie Geenan, G. J. MacFarlane, Eco J C de Geus, Johannes H. Smit, Brenda W J H Penninx, Joost Dekker: “Dysregulation of the autonomic nervous system and its association with the presence and intensity of chronic widespread pain”, Arthritis care & research 64, no. 8 (2012): 1209-1216)), low cardiovascular baroreflex sensitivity system (Chung O Y et. al, “Baroreflex sensitivity associated hypoalgesia in healthy states is altered by chronic pain”, Pain 2008, 138(1): 87-97), immunological high pro-inflammatory cytokines (Edwards R R et. al: “Association of Catastrophizing with Interleukin-6 Responses to Acute Pain”, 2008; 140(1): 135-144, doi: 10.1016/j. pain. 2008.07.024) and neurologically cognitive-affective dysregulation (Hart R P et. al: “Chronic pain and neuropsychological functioning.” Neuropsychol. Rev. 2000; 10: 131-49). An alteration in one, some or all of these conditions may incidentally be used for monitoring the efficacy of the relevant treatment of the chronic long term pain condition. This is implemented by one of the embodiments of the device according to the present invention.

The societal cost of long term pain alone is higher than that of cancer and diabetes combined¹. However, discovering and implementing a satisfactory treatment for long term pain has proven to be difficult. This is partly due to the complexity of pain etiology, which has created a wealth of diagnoses and treatments. Pain is by definition a complex phenomenon, including both sensory-discriminative, motivational-affective and cognitive-behavioral factors^{2 3}. The International Association for the Study of Pain (IASP) recognizes the complexity of pain etiology by defining long term pain as “any unpleasant sensory and emotional experience associated with actual or potential tissue damage, or described in terms of such damage”, lasting for more than three months⁴. Examples of long term pain are fibromyalgia syndrome, migraine, healed or non-healed wounds from traumatic events, the effect of diseases such as cancer, nerve damage, muscle or tissue damage, pain from tissue graft, pain from rheumatic disease or from bone surgery, etc. Pain may also originate from “imagined” states, i.e. states where the pain sensation does not originate from locations with physical damage. Such pain sensations may include “ghost” pain being transmitted by nerve paths originating from a limb that has been removed or is missing. Long term pain may even originate from treatment strategies of diseases such as chemotherapy in cancer treatment. Within the ambit of “pain” may even come states such as long-term nausea, e.g. sea sickness, or long-lasting mental states such as depression, anxiety or even withdrawal symptoms when being cured of drug-addiction, either voluntarily or involuntarily. Also states such as withdrawal symptoms from tobacco addiction or alcohol addiction may be alleviated by using the device according to the present invention.

Recently, a new classification system for pain was suggested for the upcoming International Classification of Disorders (ICD)-11. This new issue will categorize a large group of pain patients as suffering from widespread chronic primary pain. In Norway, the HUNT project reported a chronic widespread pain prevalence of 17% in the general population. More importantly, 53% of these also reported chronic widespread pain ten years later, which indicates that many Norwegians suffer from such states persistently⁵. The new widespread chronic primary pain will also include patients diagnosed with fibromyalgia syndrome⁶, and this is a pain condition that is not effectively treated solely by analgesics⁷. This has prompted basic research into the pathogenesis of widespread chronic primary pain in an attempt to find more effective treatments. Improved therapeutic alternatives for this population are highly warranted and would mean a great deal to both the individual patient and the (Norwegian) society as a whole.

Proposed Etiology

Hypotheses for what causes widespread chronic primary pain include, but are not limited to, immunological responses and the role of pro-inflammatory cytokines, autonomic dysregulation, and psychobiological origin related to major depression or cognitive-attentional syndromes⁸.

Much of the basic research addressing etiology of widespread chronic primary pain is focused on fibro myalgia pain. While the role of neurogenic inflammatory responses in fibro myalgia is debatable, multiple studies have shown markers of inflammation in patients diagnosed with fibro myalgia disease, including high levels of C-reactive protein and pro-inflammatory cytokines^{9,10}.

In some cases inflammation plays an important role in pain and can even induce pain on its own without the presence of tissue damage. There is evidence to support that fibro myalgia is associated with immune dysregulation of circulatory levels of pro-inflammatory cytokines. This affects the neural dysfunction of pain-related neurotransmitters such as substance P^{11,12}.

Increased neurogenic inflammation is also reported in fibro myalgia. Interleukin (IL)-8 levels are higher in fibro myalgia patients than control cases¹². Depending on their concentration, cytokines like IL-8 may induce symptoms such as fatigue, fever, insomnia, pain, and myalgia¹³.

Noradrenaline is a hormone regulating stress-reactions, blood pressure, depression and inflammations. The use of the device according to the present invention also has a beneficial effect on the levels of noradrenaline through its effect on the vagus nerve.

Another potential etiological factor lies within the autonomic nervous system. The autonomic nervous system is a hierarchically controlled brain body nexus that integrates the external environment with the internal milieu to maintain homeostasis. It is composed of two broadly opposing branches: the sympathetic nervous system and parasympathetic nervous system. Elevated sympathetic activity increases muscle tension and impairs local microcirculation, possibly causing painful ischemia. Nociception-induced and sympathetically maintained vasoconstriction leads to insufficient blood flow for working muscles, which in turn can trigger muscle nociception¹⁴. Consequently, in one embodiment of the treatment of chronic pain using the device according to the present invention, the treated individual is placed in a relaxing environment (e.g. with soothing music, pleasant temperature, incense, soothing massage, etc.) and may or may not be administered relaxing substances (e.g. beverages such as tea or other appropriate relaxing substances).

The lack of a clear pathogenesis in widespread chronic primary pain has also led to psychobiological models being advocated as explanations for the development and maintenance of symptoms. One such hypothesis¹⁵ states that participants with complex and unspecific health complaints tend to worry and obsess over their situation, directing their attention to their problems while keeping a sustained, cognitive activation of physiological stress responses. Bodily signals may then acquire an increased value and increased attention to the extent that normal somatic feedback becomes an overwhelming somatic experience, manifesting as muscle pain and fatigue¹⁶.

Heart Rate Variability and Widespread Chronic Primary Pain:

Heart rate variability is the variability of the intervals between successive heart beats and is regarded as a predictor of the capacity to regulate physiology and emotional responses to internal and external influence¹⁷. This is particularly true for high-frequency heart rate variability, which correlates with the functionality of the parasympathetic nerve system¹⁸. Heart rate variability is thus a physiological reflection of both descending cognitive-affective factors and ascending baroreceptor activity during respiration¹⁹. A balance between the excitatory sympathetic nerve system and inhibitory parasympathetic nerve system is required to adaptively respond to external stimuli. Practically, this means that a high heart rate value indicates a highly adaptable nervous system, while low heart rate value indicates lower physiological reactivity. Low physiological reactivity has been associated with a wealth of chronic health problems including widespread pain¹⁷.

Dysregulation of the autonomic nervous system and reduced heart rate variability have also been implicated in the pathogenesis of fibro myalgia m. A recent meta-analysis concluded that patients with chronic pain have decreased parasympathetic activation measured by heart rate variation when compared to healthy controls, and this effect is largely observed in studies on fibromyalgia¹⁷. A study on war veterans with medically unexplained symptoms adds to this by showing heart rate variability being inversely correlated with pain²¹.

Available Research Indicates that there are Two Trajectories that Connect Low Heart Rate Variability with Widespread Chronic Primary Pain¹⁷:

Low parasympathetic tone may place an individual at greater risk of chronic pain because of impaired ascending capacity to regulate noxious signals within the central nervous system. For the purpose of the present disclosure, this is defined as trajectory 1 (T₁). Alternatively, the experience of long-term pain may influence the autonomic nervous system through altered brain activity in structures involved in the descending inhibitory modulation of pain. For the purpose of the present disclosure, this is defined as trajectory 2 (T₂).

Manipulating T₁: Contemplative Pain Treatment Program: Focused Deep Breathing

Due to the resiliency of widespread chronic primary pain and the lack of effective pharmacotherapy treatment, patients commonly tend to seek alternative treatments to great expense and little effect²². One of the most popular forms of alternative care provided to patients complaining about widespread chronic primary pain is mindfulness-based stress reduction activities, which has shown effects for a wide variety of medical conditions. However, mindfulness-based stress reduction effects are highly variable and unreliable for a broad range of long term pain conditions and has proven to be particularly ineffective when it comes to lowering pain and psychological distress in fibro myalgia patients²³. A recent systematic review and meta-analysis concluded that there is limited evidence to support any type of effectiveness of mindfulness-based interventions alone for patients with widespread chronic primary pain²⁴.

The fact that mindfulness-based stress reduction activities alone show to be clinically unreliable at treating widespread chronic primary pain stands as a strong justification as to why researchers in the fields of pain management and care have a need to explore new and innovative forms of integrative mind-body treatments that can target and modulate specific neurophysiological systems that are uniquely hindered in patients with widespread chronic primary pain. This is one of the reasons why there exists a need for alternative treatment plans or devices for treating widespread chronic primary pain optionally in combination with mindfulness-based stress reduction. As indicated supra, a method for reducing muscle-tension is one of the options for improving the pain treatment and pain reduction with the device according to the present invention by stimulating the vagus nerve. One of the possible methods for reducing muscle-tension is by practicing mindfulness-based stress reduction techniques, and this may in one embodiment be combined with the use of the device according to the present invention for treating widespread chronic primary pain conditions. The phrase “widespread” in the present context relates primarily to the sensation of pain in more than one location in the body of the relevant individual, said locations being removed from each other, and possibly affecting more than one site or organ in the body.

The neurophysiological systems that plays a large role in the successful modulation and care for long term pain and which will be targeted for a combination treatment together with the use of the device according to the present invention, are the long term pain matrix, the vagus nerve, and the parasympathetic nervous system. In order to target these specific systems, the device according to the present invention may be used in a new and innovative meditative-based treatment technique called the contemplative pain treatment program which allows patients to engage both cognitively and effectively with their pain while utilizing particular deep breathing techniques that have shown to increase vagal and parasympathetic activity.

In order to optimize the contemplative breath awareness techniques in the contemplative pain treatment program the proposed method will guide patients in deep breathing as defined by The National Institutes of Health National Center for Complementary and Integrative Health²⁵. By training patients in deep breathing with a focused attention it will be possible to not only modulate cognitive and affective pain-associated neurological systems but also modulate the activity of two physiological systems associated with the modulation of high frequency heart rate variability: vagal nerve activity and parasympathetic nervous system activity¹⁷. Highly relevant for the current invention is a finding that sham-controlled vagus nerve stimulation modulation of inflammatory reaction to endotoxins failed to produce any reduction in plasma levels of inflammatory markers or self-reported symptoms. This indicates separate mechanisms influencing inflammatory processes in parasympathetic manipulation through breathing and vagus nerve stimulation²⁶.

In many situations one of the external markers for pain in an individual is that the breathing practiced by the individual changes from normal breathing with semi-slow (one inhalation per second) deep inhalations (filling at least half of the longue volume with air) to a short and shallow breathing pattern. Such a pain-related breathing pattern my be involuntary, but may be influenced by the individual by consciously taking control of this breathing pattern and bringing it back to a more normal breathing pattern.

In one proposed manipulation of T₁, the contemplative pain treatment program intervention will make use of multiple determined breathing techniques which utilizes diaphragmatic breathing.

Diaphragmatic breathing is a process of breathing by which subjects bring their attention to their breath and slowly direct each inhalation into the abdomen, allowing the abdomen to gently expand with inhalation and release back toward the spine on the exhalation. In this way, patients learn to activate and tone diaphragmatic muscles and free tension from the abdominal area while simultaneously optimizing lung capacity, gas exchange, and blood-oxygen content²⁷. This technique may be coupled with a contemplative breath-awareness practice, optionally relegated to awareness or compassion, in order to modulate both cognitive and affective systems inherent in pain perception.

Breathing as a form of therapy or treatment has shown to be quite beneficial for a wide variety of psychophysiological conditions. In particular, patients diagnosed with chronic low-back pain who underwent a six to eight week (12 session) basic breath therapy program were able to significantly lower measures of both pain perception and disability when compared to physical therapy²⁸.

Moreover, research has shown that specific yogic-based breathing techniques have had significant modulatory effects on both psychological and physiological systems. A study which conducted a spectral analysis of heart rate variability during a slow yogic breathing practice called Nadi Shodhana Pranayama (e.g. alternate nostril breathing) determined that deep breathing techniques can provide valuable insights into neurophysiological mechanisms of autonomous nervous system regulation that are usually not attainable via normal breathing. In the aforementioned study, the practitioner under analysis was able to significantly increase measures of respiratory sinus arrhythmia which indicates a strong synchronicity between heart rate variability and respiration²⁹. Respiratory sequence alteration, which can be used as an index of cardiac vagal function, increases the efficiency of pulmonary gas exchange by positively influencing efficient ventilation/perfusion matching³⁰. Another study which analyzed the effects of Nadi Shodhana Pranayama on heart rate, systolic and diastolic blood pressure, peak expiratory flow rate, and simple problem-solving ability found that 20 minutes of Nadi Shodhana Pranayama practice significantly lowered basal heart rate and systolic blood pressure and significantly improved peak expiratory flow rate, as well as the time taken for simple problem-solving following pranayama practice³¹.

Moreover, another determined breathing technique called Bhramari pranayama (which consists of humming while inhaling slowly for about 5 seconds and then exhaling slowly for about 15 seconds while keeping the ears closed with one's hands) induced a strong parasympathetic dominance on the cardiovascular system of practitioners³².

Manipulating T₂: Transcutaneous Vagus Nerve Stimulation

The vagus nerve is the 10th cranial nerve and is involved in the regulation of nearly all inner organs. Approximately 80% of the vagus nerve fibers are afferent, carrying essential information from the periphery to the brain. The parasympathetic nervous system, whose main neural substrate is the vagus nerve, exerts a fundamental anti-nociceptive role. Whereas the spinal cord conveys nociceptive information, the vagus nerve potentially influences pain through mediating the “inflammatory reflex”; a mechanism that the central nervous system utilizes in order to regulate innate and adaptive immunity³³. There are five main reasons why stimulation of the vagus nerve has been investigated in the treatment of widespread chronic primary pain³⁴.

First, the vagus nerve activates a systemic pathway, the hypothalamic-pituitary-adrenal axis, through which increased free, circulating cortisol inhibits pro-inflammatory cell proliferation³⁵. This is substantiated by experimental results showing vagal nerve activity being inversely correlated with IL-6 blood levels³⁶. Second, experimental data suggests that the vagus nerve inhibits sympathetic nerve activity. As an example of this, a vagotomy increases epinephrine levels in the adrenal medulla. Moreover, acetylcholine—the primary neurotransmitter of the vagus nerve, reduces levels of norepinephrine in the central nervous system³⁷. Third, vagal nerve activity is inversely correlated with malondialdehyde, which is a biological marker of oxidative stress³⁸. Oxidative stress contributes to inter alia fibro myalgia through reactive oxygen species but vagal nerve activity may inhibit this. Fourth, stimulating left prefrontal cortex (as the vagus nerve system does) by repetitive transcranial magnetic stimulation was found to reduce postoperative pain and suggest that vagal nerve activity may be associated with activity in the “higher order” brain regions capable of modulating pain, including the left prefrontal cortex and the PAG, as well as inhibition of the limbic region³⁹.

One possibility of pain relief through vagal nerve stimulation could also be the relief of migraine by such stimulation.

### PRIOR ART

Traditionally, vagus nerve stimulation has been administered through invasive procedures such as acupuncture or surgical implantation of electrodes around the cervical vagus nerve. These electrodes are then connected to a stimulating device implanted under the anterior chest wall. However, such procedures entail a risk for adverse events. If there are no complications from surgery, the most frequently reported adverse events include voice alteration, paresthesia, cough, headache, dyspnea, pharyngitis and pain at the site of stimulation⁴⁰. These adverse events often require a decrease in stimulation strength or permanent deactivation of the device.

However, newer non-invasive vagus nerve stimulation delivery systems do not require surgery and allow users to change the degree of stimulation when needed.

These non-invasive systems improve the safety and tolerability of vagus nerve stimulation, making it far more accessible and feasible. One such non-invasive system, Transcutaneous Vagus Nerve Stimulation has been tested experimentally and has been shown to reduce evoked pain⁴¹, and decrease somatic pain sensitivity in healthy participants⁴². The preferred transcutaneous vagus nerve stimulation system according to the invention uses in one embodiment the auricular branch of the vagus nerve, which extends to the concha of the outer ear⁴³. This means that the nerve branch can be stimulated through the skin (transcutaneous) with electrical impulses. Intensity, pulse duration and frequency of the transcutaneous vagus nerve stimulation have been adjusted to induce signaling from the A-β fibers of this auricular branch⁴⁴. Like the cervical branch of the vagus nerve, these fibers project to the nucleus tractus solitaries, raphe magnus, locus ceruleus, amygdala and periaqueductal gray, which are systems involved in the descending inhibitory modulation of pain. Data from experiments show reduced pain sensitivity towards mechanical stimuli and reduced temporal summation of noxious tonic heat. Exaggerated temporal summation of pain characterizes several chronic pain disorders, suggesting that mechanisms for temporal summation may be involved in the onset or maintenance of widespread chronic primary pain. The collected data suggest that transcutaneous vagus nerve stimulation may be a target for manipulation of T₂.

### SUMMARY OF THE INVENTION

It is an object of the present invention to provide a device for treating long-term or chronic pain in an individual through vagus nerve stimulation, and which is improved above prior art devices and methods for this purpose. This object is achieved in a device as disclosed in the appended claims.

In particular, the present invention includes a device stimulating the vagus nerve by using electric pulses which are adapted to the particular type of pain. Further, the device includes sensors for monitoring the effect of the treatment, and which may be used as a feedback to adjust the pulses for obtaining an optimum effect on the individual.

### BRIEF DESCRIPTION OF THE DRAWINGS

The construction and function of the device according to the present invention will be better understood by reference to the enclosed figures wherein:

FIG. 1 depicts an embodiment of possible locations for the electrodes on a human ear associated with the use of the device according to the invention.

FIG. 2 depicts an embodiment of alternative locations for the electrodes on a human ear associated with the use of the device according to the invention.

FIG. 3 depicts the electrodes of an embodiment of the device according to the invention constructed as a clamp.

FIG. 4 depicts the electrodes of an embodiment of the device according to the invention constructed as a hoop, crook or bow encircling the ear.

FIG. 5 depicts a division system of the ear for reference to areas of the external ear concha to target when using the device according to the invention to alleviate or treat LT pain.

FIG. 6 depicts the sections of the ear and their names.

FIG. 7 depicts an embodiment of an ear piece for stimulating the vagus nerve in the ear.

FIG. 8 depicts an embodiment of a particular construction of an ear piece for stimulating the vagus nerve in the ear, the device in this embodiment carrying its own power source.

FIG. 9 depicts an embodiment of a particular construction of an ear piece for stimulating the vagus nerve in the ear, this device being meant to be connected to an external power source.

FIGS. 10 a-c depict examples of different locations to place ear pieces of a device according to the present for embodiments of such ear pieces.

### DETAILED DESCRIPTION

As is shown in the figures the device according to the invention comprises a source for electricity I shown as a battery assembly, alternatively coupled in parallel or series or both for creating a voltage within the interval 1.0-50,000 V and a current within the interval 0-20 mA and preferably an amperage within the interval 0-15 milliamperes, more preferred 0-10 milliamperes, more preferred 0-8 milliamperes, e.g. 1, 2, 3, 4, 5, 6, 7 or 8 milliamperes. In the context of the present invention the source of electricity may in principle be any source of electricity such as a solar-powered battery or any battery assembly, an electricity outlet for domestic or industrial electricity (110-220 V) combined with one or more devices for bringing the voltage and amperage of the device to lie within the above indicated intervals. Devices for transforming electrical energy into the indicated intervals are known to the person skilled in the art.

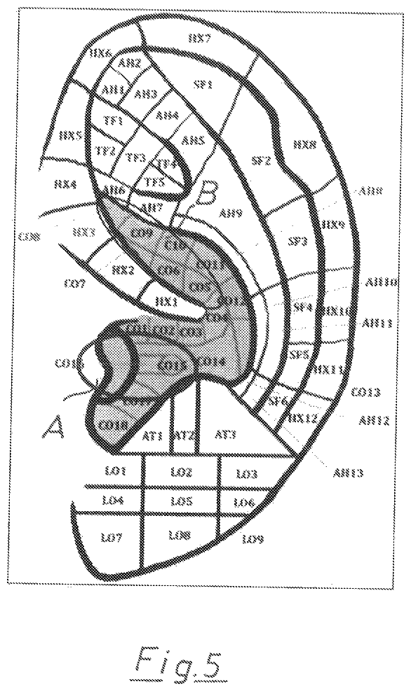

As indicated in FIG. 5 the external ear is sectioned into areas L01-L09; AT1-AT3; AH1-AH13; CO1-CO19; HX1-HX12; SF1-SF6 and TF1-TF5. Electrical stimulation of the external ear for alleviating or treating pain sensations through stimulation of the vagus nerve is complicated by multiple neural communications CO6, CO9, CO10 being connected to the Trimegenius nerve and CO8 being associated to the occipital nerve. However, it is the strongest connection to LC fMRI studies suggest the inner tragus as an option for CO15, CO16. The stimulation for treating or alleviating pain with the device according to the present invention is preferably performed by any one or a combination of or all of the areas CO5, CO6, CO9, CO10, CO11, CO12, AH6 and AH7 as well as the areas COCO1, CO2, CO14, CO15, CO16 and CO17, although any of the areas in the CO section as well as the HX1, HX2, HX3 HX4 sections or any combination thereof may be stimulated as well.

In FIG. 5 there are indicated areas A and B comprising sections CO1, CO2, CO14, CO15, CO16 and CO17 for section A and CO5, CO6, CO9, CO10, CO11, CO12 and parts of HX1, HX2, HX3, HX4, AH6 and AH7 of the external ear giving particularly improved pain relief when treated with the device according to the present invention as indicated infra.

Technical details pertaining to the preferred electrical stimulations as well as the conditions affected by the stimulations are indicated in Table 1.

TABLE 1

| Pulse 0-1000 Hz 0-30 | Time (min) | Condition | mA up to 8 |
|---|---|---|---|
| 250-500 25 Hz | 60 | Pain | up to 8 |
| 250 100 Hz | 45 | Pain | up to 8 |
| 250 10 Hz | 45 | Arthritis | up to 8 |
| 250-1000, 25, 134 Hz | 45 | Migraine | up to 8 |
| 250 30 Hz | 60 | IBS | up to 8 |

Referring to the figures the electricity from the power source I is in one embodiment conducted by wires 1,2 to at least one pair of electrodes 3,3′,4 for creating a voltage difference between said electrodes when connected to the power source I. The electrodes 3,3′,4 are constructed to fit inside the ear 5 of the treated individual for affecting the vagus nerve by manipulating the voltage of the device.

The electrodes 3,3′,4 may in an alternative embodiment be made of resilient or formable material such as a soft metal or metal alloy or a resilient or formable covering or sheath coating said electrodes or electrode wires 1,2,3,3′,4 for easing their correct location in the ear 5. Examples of such materials are waxes, resilient polymers (plastic, rubber or mixtures thereof). Alternatively the electrodes 3,3′,4 may be carried by a brace, a hook, a crook or a bow 6 to be placed around or on top of the ear 5. Such items may be used for ensuring the correct placement of the electrodes 3,3′,4 inside the ear 5 or touching the concha in the areas indicated supra. In an alternative embodiment the electrodes 3,3′,4 may be constructed as patches or tags optionally equipped with a sticky substance for ensuring the correct placement of the electrodes 3,3′,4 in the ear as well as ensuring their permanent or semi-permanent placement in the ear (at least as long as the electrical stimulation is ongoing). The electrodes 3,3′,4 may also be part of a clamp or clamp assembly 7 for clamping the electrodes 3,3′,4 to the ear.

The ear piece may also carry its own power source (battery) and may in certain embodiments also carry its own programing device 8 for adjusting the parameters of the electrical stimulation of the ear.

The parts of the electrodes 3,3′,4 that are in contact with the skin of the ear 5 are preferably not electrically isolated for providing a good electrical contact between the electrodes 3,3′,4 and the ear 5. In an alternative embodiment the contact between the electrodes 3,3′,4 and the ear 5 may be enhanced by covering the skin of the ear 5 beneath the electrodes 3,3′,4 and/or the surface of the electrodes 3,3′,4 located in contact with the skin of the ear 5 with an electrically conducting slave or coating ensuring an optimal stimulation of the vagus nerve through the ear.

In the figures there is shown an attachment of the electrodes 3,3′,4 of the device according to the invention to a left human ear 5, but a right human ear is equally effective. It is also possible to stimulate both ears simultaneously, or optionally one ear at a time, or optionally alternating the stimulation of the ears 5 or other body parts carrying the vagus nerve of the treated organism or patient.

The device according to the invention may be used in both human and veterinary pain reduction procedures.

In a veterinary setting the treatment of an organism may use an equivalent site as the relevant sites in the human ear (see supra) or may be located in other parts of the body. The pain treatment or enhancement of the immune system by stimulating the vagus nerve in both humans and in animals of may be conducted as disclosed. It is preferred that the present treatment program is performed on mammals at various sites on the mammalian body. Such sites may be located in the paws, abdomen or neck/throat as well as in the ear of the animal. The same voltage and amperage intervals as well as the electrical pulse intervals and pulse/stimulation frequencies as indicated supra for the human ear may be used. An animal in distress and/or pain show many of the same symptoms as a human (hart rate variations, breathing variations, external distress markers (body posture, muscle tension, whimpering or other sound issuings, etc.), indicating to a veterinary that administration of pain relief by using the device according to the present invention is in order. The reduction of halting of such markers may be an indication that the treatment is effective. Treatments may be conducted by one up to several stimulations per day, and may be part of a pain treatment program or treatment strategy lasting over a period of time such as for days, weeks, months or even years.

One of the challenges for securing the activation of the vagus nerve in the ear, is to construct a device to fit in the ear and to contact the indicated areas thereof. This may be accomplished in numerous ways, whereof some will be illustrated infra.

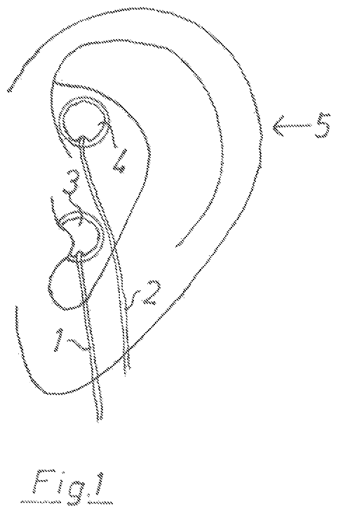

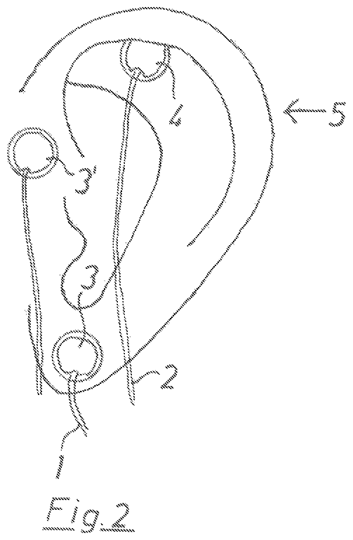

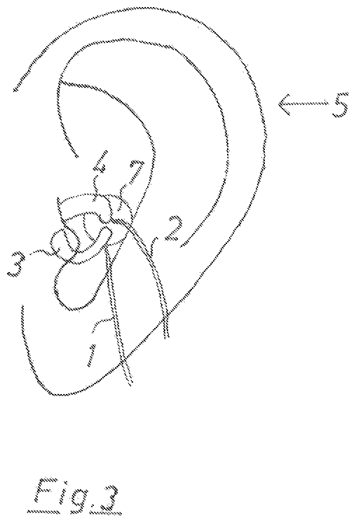

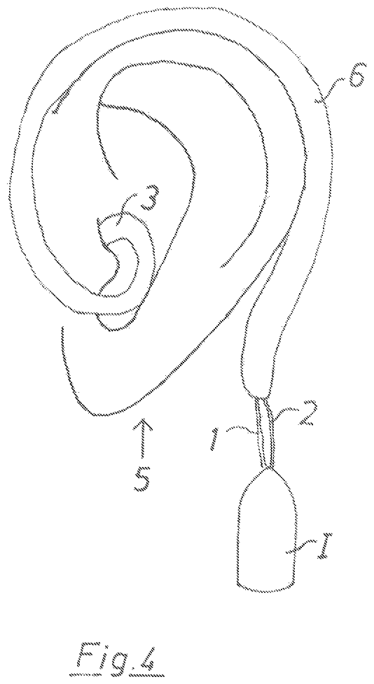

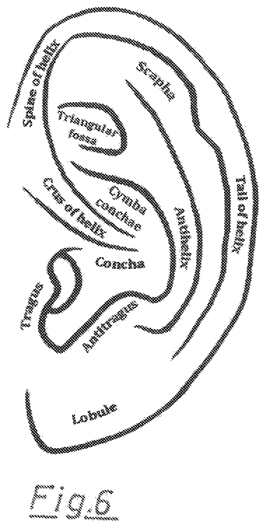

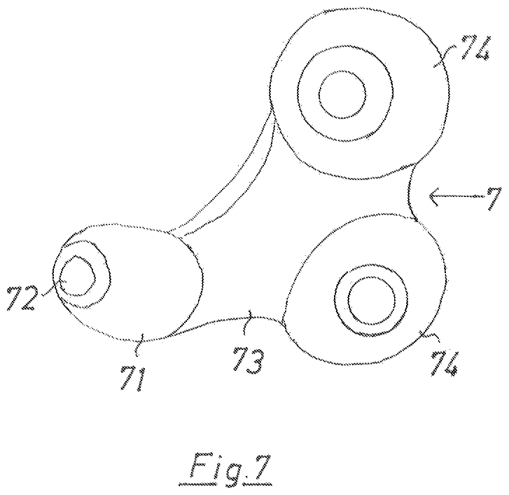

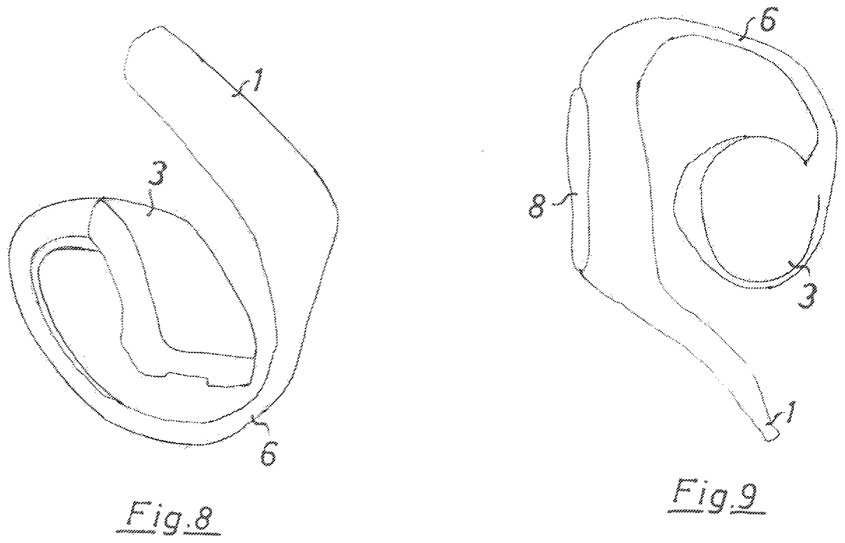

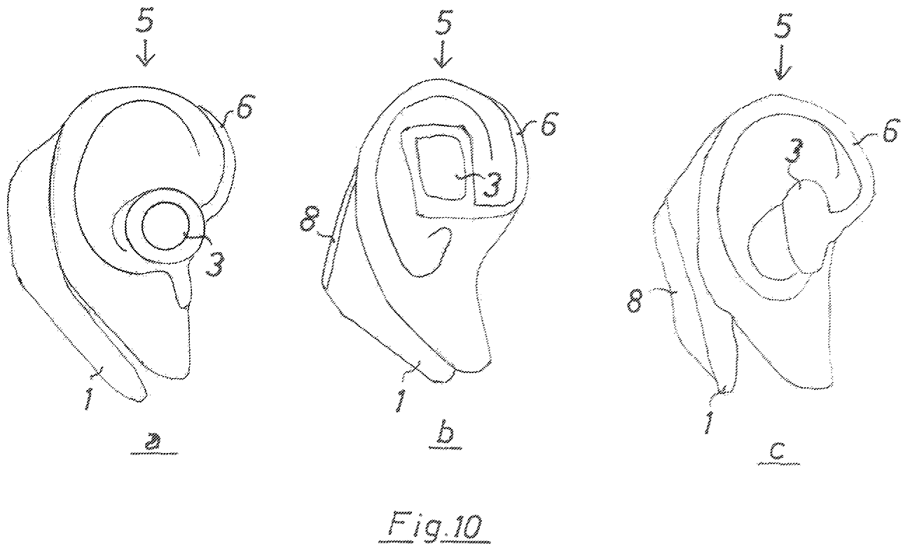

With reference to FIGS. 5, and 6 depicting a human ear, it is illustrated here that the areas to stimulate electrically with the device according to the invention are sections A and B indicated in FIG. 5, largely coinciding with the concha and cymba conchae of FIG. 6. Opposite the concha area the tragus is located forming a tract into the inner ear. The cymba conchae form normally a ledge of the ear in the transition zone into the antihelix. One of the challenges in structuring and constructing the ear device of the present invention, is firstly that the ear device preferably is to lie continuously in the ear during its use, which may be for days or weeks when treating chronic or long term pain and associated states. The ear device should also preferably be easy to insert and should also be comfortable to wear, while simultaneously providing electrical stimulation to the vagus nerve in the ear. In FIGS. 1-4 and 7 there are suggested certain embodiments of the ear section of the device ensuring electrical stimulation of the vagus nerve in the ear. The structure of the embodiment of the ear piece 70 shown in FIG. 7 includes one mainly cylindrical ear piece 71 fitting into the ear opening or canal and including a section 72 establishing electrical connection between the skin of the ear and the ear piece 70. The ear piece section 72 fitting into the ear canal is located on a foundation 73 and cooperates with at least one mainly cylindrical protrusion 74 fitting inside or against the antihelix of the ear for securing the ear piece of this embodiment to the ear without any need for adhesives or clamps or the like. The cylindrical pieces 71, 74 are constructed or rest against the skin of the ear thereby establishing an electrical connection sending the disclosed electrical pulses through at least one of the areas A and B indicated in FIG. 5.

For convenience the batteries (not shown) may be located or kept in a case or holder I, and the wires 1,2 leading from the holder I to the electrodes 3,3′4 may be equipped with a switch (not shown) for turning the device on or off. The device may in certain embodiments be controlled or programed by a programing console 8 located externally or carried by the device. Individual and personal programing of the electrical stimulus provided by the device according to the present invention may be done by each individual or by a doctor/therapist for establishing a treatment program or treatment procedures.

In normal circumstances it is sufficient to place the active device in one ear only or at one location for activating the vagus nerve. However, it is also possible to place the electrodes 3,3′,4 on several locations simultaneously for ensuring the activation of the vagus nerve. It is also possible to influence the vagus nerve by stimulation through both ears alternately or simultaneously.

The embodiment of the ear piece shown in FIG. 7 includes mainly cylindrical pieces 71,74 conducting the electrical pulses to the ear, secured to a foundation 73. The foundation 73 is preferably semi-pliable or pliable for molding the ear piece 70 to fit the anatomy of the relevant ear. The material of this foundation may be a polymer such as a wax material or a rubber material or a plastic material. The foundation 70 could in one embodiment include electrical connectors or sockets and possibly electrical wires (not shown) for transporting the electrical signals or pulses to the mainly cylindrical pieces 71,74. The cylindrical ear pieces 71,74 may alternately and optionally be electrically connected to batteries (not shown) located in the foundation 73, and the activation of the ear device may be performed by an external control device (not shown).

The device according to the present invention may be used in professional as well as in private settings. As mentioned supra, the device according to the invention may be used in combination with alternative techniques for reducing the chronic or long-term pain, such as meditative relaxing practices or optionally combined with other types of pain reduction or pain-killing remedies, e.g. medications or acupuncture.

In other circumstances the device according to the invention may be used for reducing pain experienced by the ingestion or infusion of pain-inducing substances such as chemotherapeutics, e.g. in cancer-treatment procedures.

It is also used for collecting and recording health data through sensors integrated into the device. Users can track, monitor and privately own their health data and optimize vitality. A software application will act as a platform where users can share or sell their data to third parties. This software will involve artificial intelligence to analyze health data and blockchain technology to securely share this data.

The device further comprising sensors for monitoring and recording health measurements in order to track and optimize daily health.

The sensors may comprise at least one of biosensors, EEG (Electroencephalography) sensors/electrodes, Photoplethysmogram, Pulse oximeter, Electrocardiogram, infrared sensors, temperature sensors, and/or humidity sensors, wherein the sensors are integrated in the device with electrodes that send data signals to a software application on a smartphone/device that collects, records and analyzes health measurements with the help of algorithms and artificial intelligence. This application acts as a platform for users to share or sell their privately owned data to third parties through blockchain technology.

Sensors give feedback automatically through artificial intelligence which then adjusts the device accordingly to its greatest efficacy for the individual.

The electrical current fed to the electrodes 3,3′, 4; 72, 74 may be of a constant or alternating type. In a constant administration of electricity to the electrode(s) 3,3′,4; 72,74 the polarity of the current is not varied, although it is in one embodiment possible to vary the polarity of the electrical current. In both the alternating current embodiment as well as the direct current embodiments it is possible to send the current in current trains for activating the vagus nerve. In such electrical current trains the variation of the current peaks in the current train may lie within the interval 1 to 1,000 ms. Current trains may also be the same or may alternatively be different from each other, and the time interval between current peaks in the current trains may also be the same or be varied. The variation between current peaks may lie within the interval 1 to 1,000 ms. The frequency of the electrical stimulation signals may in one embodiment lie within the interval 0-50,000 Hz such as 10-500 Hz, e.g. around 20-100 Hz.

Impact on Patient Care and Other Strategic Considerations

The device and use thereof according to the present invention may improve health care for widespread chronic primary pain sufferers nationally as well as globally; the device and use can be immediately implemented in clinical care through the dissemination of breathing techniques and commercialized transcutaneous vagus nerve stimulation apparatus.

Widespread chronic primary pain is one of the most challenging health problems globally: Prevalence is high, disability is severe, treatment options are limited, and there is a strong negative impact upon employment issues, social security systems, family networks. On 2 Mar. 2015 the Norwegian National Council for Priority Setting in Health Care advised that research on widespread chronic primary pain should be prioritized. Moreover, the new guidelines from Department of Health on pain management specifically emphasized the lack of knowledge in these patients. At present, the health care system experiences a very palpable lack of treatments in the encounter with widespread chronic primary pain. The results obtained from the device according to the present invention and the use thereof can be immediately implemented in clinical care nationwide as well as globally, and will potentially provide a most needed tool for healthcare professionals. The transcutaneous vagus nerve stimulation stimulators according to the invention may be made available for purchase and can easily be provided to primary care physicians. They could then be a first line offer of treatment in patients with widespread chronic primary pain. Moreover, since breathing techniques have been specifically developed and optimized for the treatment of chronic pain by taking into consideration long term pain matrix data, parasympathetic nerve system activity, and vagal tone, one could expect an even greater clinical effect upon pain patient outcomes. The combination of the device according to the invention with breathing techniques of the kind indicated supra, may be implemented with an open access protocol, making the breathing technique available for primary care physicians.

Gender issues: Widespread chronic primary pain is far more common among females than males, the ratio being about 3:1. Thus, it is a typical ‘woman's disease’, with strong impact on women's health, but traditionally receiving little attention scientifically and clinically.

Previous research experiences: The study associated with the device and use thereof according to the present invention is integrated in the Norwegian Pain Association as well as the National organization for fibro myalgia patients, lending strong support in relevant environments.

Translational aspects: The potential applications of the device and use thereof according to the present invention are enticing. The training and stimulation is possible to deliver to most patients with widespread chronic primary pain. A single well-performed project could here provide valuable answers detailing the role of parasympathetic manipulation and autonomic activation in a large group of pain patients who have an unclear etiology and lack effective treatment options. Thus the device and use thereof could be valuable in diagnostic settings as well as therapeutic settings.

In a possible embodiment of the device and use thereof according to the present invention, it is possible to equip the ear-contacting sections 3,3′,4; 71,74 with devices for monitoring the state of the organism during and/or after the vagus nerve stimulation. Devices for monitoring the heart rate/heart beat frequency, the blood pressure, the oxygen uptake in the blood, the temperature etc. are known and may be implemented into the ear-connecting sections 3,3′,4; 71,74 of the device according to the invention. Also it may be possible to monitor external indicators of pain/pain relief such as muscle tone/muscle relaxation, body stature, oral accounts from the patient, etc. to determine the efficacy of the treatment using the device according to the present invention. Data from such monitoring may be collected and used for verifying the optimal pain relief in cases where the device according to the invention is used. Such data may also be compiled with data for pain relief using other pain relief regimens such as medical pain killers, massage therapy, muscle relaxants, etc. Such collected data may even be compounded or compiled into a data collection for inspection and possibly for creating a treatment protocol or regimen both generally and individually. Such data may include a comparison between treatment parameters (location of vagus nerve stimulation, duration of vagus nerve stimulation, parameters of electrical stimulation, etc.) and the success of such treatment (improvement of the bodily parameters indicated supra).

In various embodiments the collection of data may be performed by monitoring the HRV (heart rate variation) before and during the treatment with the device according to the invention. The monitoring of the state of the treated individual may also be performed by using an accelerometer. States such as temperature and humidity of the skin may also be included in the collection of data.

Since affecting the vagus nerve has a positive influence on the well-being of the treated individual, one possible use of the device according to the invention may be to allow peak performance in training and competitions for athletes. This particular use includes the relevant athlete using the device over a training period or competition period. Since affecting the vagus nerve influences the well-being of the entire body, influencing the vagus nerve in training settings will improve the effect of the regular training of the athlete. This improvement will influence both the muscular and the emotional/nervous state of the organism leaving the organism in a better state to assimilate the training effects.

Another example of the influence of the state of the vagus nerve on the state of the organism is that a person treated with the device according to the invention will enter into a more defensive immune state than a non-treated individual. Such an enhanced immune state will provide a better chance to combat both bacterial and viral infections. It has been shown that electrical stimulation of the vagus nerve using vagus nerve stimulation can improve the body's natural ability to regulate the inflammatory response and may be potent enough to suppress pro-inflammatory cytokines and prevent death from COVID19 infection, particularly if used early enough in the course of infection e.g. during the course of hospitalization. Viral infections to be treated with the device according to the invention include both retroviral infections as well as infections with normal viruses. As examples of viral infections to be treated with the device according to the present invention there may be mentioned covid19 infections, SARS infections, bird influenza, swine influenza and AIDS infections. Bacterial infections such as inflammations may also be treated by using the device according to the present invention to place the immune system in a more alert state.

As mentioned supra the influence of the vagus nerve with the device according to the present invention may be combined with other stimulations as well. One particular external stimulus type that has proven to be effective for putting the organism in a receptive state, is be using virtual reality (VR) glasses and/or sound headphones on the relevant individual while stimulating the vagus nerve with the device according to the invention. To the extent that the VR glasses and/or headphones show situations of a pleasant nature to the treated individual, the use of the device according to the invention show better results than results with such VR glasses and or headphones alone or with using the device according to the invention alone. In the case of using sound headphones during the relevant treatment, it should be made certain that the sound headphones do not interfere with the ear device according to the invention. In an alternative embodiment an earphone may be integrated in the ear stimulating device according to the present invention.

The same type of results is observed when stimulating the vagus nerve in animals with corresponding electricity signals as disclosed supra for humans. However, the location for the vagus nerve stimulation may be different in animals than in humans. Such alternative stimulation locations may be stimulating the vagus nerve through electrical impulses on the neck and/or on the abdomen of the individual.

Objectives and Goals:

The device and use thereof according to the present invention aims in one embodiment to investigate the efficacy of a Contemplative Pain Therapy Program (CPTP) and Transcutaneous Vagus Nerve Stimulation on electro cardiogram results measured heart rate variation, utilizing randomized control trials with both inactive and active control conditions. Moreover, it will attempt to identify conditioned pain modulation driving a potential change. These aims will be investigated through the testing of four indicators:
H₁: It is found that by manipulating T₁ through a standardized CPT program, the vagal parasympathetic tone will be significantly increased in participants with widespread chronic primary pain measured through high frequency electro cardiogram measured heart rate variation, when compared to an inactive control condition.
H₂: It is found that by manipulating T₂ through a standardized transcutaneous vagal nerve stimulation procedure, participants with widespread chronic primary pain will significantly increase their descending pain inhibition as measured through high frequency electro cardiogram measured heart rate variation, when compared to an inactive control condition.
H₃: It is found that when testing manipulations of T₁ versus T₂ in an active control condition, there are found significant differences in electrocardiogram measured heart rate variation following the intervention which details the most effective trajectory.
H₄: It is found a significant increase in conditioned pain modulation from the widespread chronic primary pain patients showing effects on electrocardiogram measured heart rate variation.

Feasibility/Effectiveness of Device and Use

Study Design, Choice of Methodology and Analyses

Design Overview

This study will use a randomized, controlled, two-stage experimental design. This design will be implemented with six groups of LT pain patients diagnosed with widespread chronic primary pain. The two stages consist of: Stage one, where four groups of consenting widespread chronic primary pain patients will be recruited and randomized. Half of the participants will be randomized to either an experimental transcutaneous vagus nerve stimulation group or a waiting-list control group. The other half will be randomized to either an experimental CPTP-group or a waiting-list control group. This will give two randomized controlled experiments in the first intervention stage, testing H₁ and H₂. An untouched control group will also be included to control for any Hawthorne effects.

In the second stage, the transcutaneous vagus nerve stimulation treatment and the CPT program are implemented in two experimental groups, where they will serve as an active control for each other. Hence, the first intervention stage establishes potential effect for the transcutaneous vagal nerve stimulation and the CPT program. The second stage tests whether one manipulation is superior to another, testing H₃. After completion of the second stage, all blood and hair samples will be analyzed collectively, testing H₄.

Recruitment, Inclusion and Exclusion

Participants will be consecutively recruited from the clinic for Pain Management and Research at OUS, UllevÅal during 2017 August. It will be recruited and randomized a total of 80 patients with widespread chronic primary pain. The first stage of the design/investigation will include half of these patients (40) and the second stage will require the rest (40). The clinic treats approximately 1000 new patients annually, so the feasibility for recruitment is high. An overview of the two-stage study design, participant selection and flow through the project is illustrated in illustration 1.

Procedures: The transcutaneous vagus nerve stimulator [(STV02, Cerbomed, Erlangen, Germany)] according to one embodiment of the invention comprises a small stimulation unit and a bipolar stimulation electrode placed into the left concha at the inner side of the tragus by direct contact on the skin. The electrode is then placed on an acrylic body for a comfortable fit in the pinna. Participants will receive a 60-minute stimulation once a day for 6 weeks.

For the CPT program DB techniques over 6 weeks will be utilized, one session per week, which are performed by patients in the supine position. These techniques will use the diaphragm to maximize lung capacity and optimize parasympathetic nerve stimulation activity influencing vagal tone. Diaphragmatic breathing will be implemented in three different pranayama exercises: attentive awareness, alternate nostril breathing, and ujjayi breathing (i.e. auditory breath awareness).

Adherence: The participants' adherence to the training program will be monitored electronically via questback. An 80% completion of the CPTP or transcutaneous vagal nerve stimulation will be regarded as adequate adherence in this test.

Randomization: The test will use an open list randomization as the primary outcome is a physiological marker and therefore resistant to manipulation through altered expectations.

Other areas or situations wherein a non-invasive and non-medicational treatment regimen is of benefit are within the treatment of:
Anxiety/depression,
Heart conditions
Epilepsy,
Headaches/migraine,
HRV (heart rate variations)
Inflammation,
Treatment of viral diseases of both retroviral and normal type (e.g. covid 19)
Cognition (e.g. dementia, Alzheimer's disease)
Stomach ailments (e.g. Crohn's disease, ulcer, chronic constipation, etc.)
PTSD (post-traumatic stress disorder)
Idiopathic pain,
Weight gain/reduction,
Mood swings,
Post-treatment of drug withdrawal pain/symptoms.

In the disclosure supra it has been indicated that the electrical stimulation is done by placing the electrical wires/pads on the same side of the ear for stimulating the vagus nerve via the concha of the ear, i.e. by placing the electrical poles/pads on the side of the ear facing outwards from the skull. However, it may also be possible to stimulate the vagus nerve by placing the electrical poles/pads on opposite sides of the ear, i.e. by placing one of the poles/pads on the side of the ear facing the skull and the other on the side of the ear facing away from the skull. It is preferred to place the electrical pole or pad facing the skull as close as possible to the junction area between the skull and the ear (not shown).

### EXAMPLES

Power Calculation and Sample Size Estimation

An experimental study of transcutaneous vagal nerve stimulation showed reduction on bilateral pain sensitivity, however with only one study there is no replicated or established effect. This is also the case for the CPT program. The lack of knowledge prompted the current structure, making a priori estimation of power or effect difficult. However, a power analysis using cumulative distribution indicated that a total sample of 40 people would be needed to detect large effects (d=0.8) at treatment termination in stage 2. This estimation is with 99% power using a t-test between means and a significance level of 0.05.

Investigational Program

Primary outcome: The use of the device according to the present invention is explored in association of two manipulations of high frequency heart rate variation. The root mean square of successive differences (RMSSD) will be derived from electrocardiogram recordings and serve as the primary outcome. RMSSSD values correspond to parasympathetic regulation of heartbeats. The results are analyzed through existing computational infrastructure in the pain medicine research group.

Secondary outcome: Conditioned pain modulation (CPM) is tested using the published protocol from the Tromso study (see http://tromsoundersokelsen.uit.no/tromso/). This includes comparison of data from over 10000 people, increasing the ecological value of the CPM measure.

Demographics, functional limitations and symptoms: The participant's age, gender, height, weight, relationship status, education, anxiety, depression, fatigue and insomnia is used through a standardized set of questions used in an existing electronic survey at the Department of Pain Management and Research.

Statistical Analyses

Improvement in heart rate variation from pre to post treatment is analyzed using several independent t-tests and chi-squares. The subjective responses during intervention are modeled by repeated measures and by fitting random effects models with random slopes. Random effects models are considered the gold standard when analyzing repeated measure and handling missing data.

Ethics

Participation is based upon informed consent, and thorough information will be provided orally as well as in writing to the participants. All data is treated and stored without personal identifying information, and in accordance with national directives. Approbation for this specific study is sought from the Regional Committee for Ethics in Medical Research and the Data Protection Office. The study (will be) is registered at ClinicalTrials.gov, and (will) adheres to the STROBE statement.

### Example 1

This example pertains to a male individual aged 33 afflicted with fibromyalgia having long-term pain sensation over a period of 8 years. This condition was attempted to be treated during these years with treatment regimens including oral consumption of the medications Naproxen and Tramadol.

Said individual was treated with a device according to the invention comprising a head set with an ear piece of electrodes secured to the sections secured to the sections CO5, CO6, CO9, CO10, CO11, CO12, CO15 and CO16 of the left ear and affected with electrical pulses of 30 Hz for a duration of two times 30 minutes per day (morning and evening).

The effect of this treatment resulted in complete recovery and discontinuation of the ingestion of naproxen and tramadol.

### Example 2

This example pertains to an individual (female aged 49 afflicted with fibromyalgia having long-term pain sensations over a period of 9 years).

Said individual was treated with a device according to the invention comprising headset/ear-piece with electrodes stimulating the sections (CO5, CO6, CO9, CO10, CO11, CO12 CO15 and CO16) of the left ear and affected with electrical pulses of Hz for a total duration of 60 minutes per day, 30 minutes in the morning and 30 minutes in the evening.

The effect of this treatment resulted in partial recovery and reduced medication use.

### Example 3

This example pertains to an individual female aged 24 afflicted with irritable bowel syndrome.

Said individual was treated with a device according to the invention comprising headset/ear-piece with electrodes stimulating the sections (CO5, CO6, CO9, CO10, CO11, CO12 CO15 and CO16) of the left ear and affected with electrical pulses of 5 Hz for a duration of two times 30 minutes per day (morning and evening).

The effect of this treatment resulted in a 30% improvement in the irritable bowel syndrome-symptom severity scale (IBS-SSS).

### Example 4

This example pertains to a female aged 33 afflicted with fibromyalgia having long-term pain sensations over a period of 8 years and having been treated for these sensations over the period with the medications Naproxen and Tramadol.

Said individual was treated with a device according to the invention comprising headset/ear piece with electrodes secured to the sections (CO5, CO6, CO9, CO10, CO11, CO12 CO15 and CO16) of the left ear and affected with electrical pulses of Hz for a duration of two times 30 minutes per day (morning and evening).

The effect of this treatment resulted in complete recovery and discontinued medication of Naproxen and Tramadol.

### REFERENCES

1. Vos T, Flaxman A D, Naghavi M, et al. Years lived with disability (YLDs) for 1160 sequelae of 289 diseases and injuries 1990-2010: a systematic analysis for the Global Burden of Disease Study 2010. The Lancet 2013; 380(9859):2163-96.
2. Melzack R, Casey K L. Sensory, motivational and central control determinants of pain: a new conceptual model. The skin senses 1968:423-43.
3. Turk D C, Melzack R. Handbook of pain assessment: Guilford Press 2011.
4. IASP TFoT. Part III: Pain Terms, A Current List with Definitions and Notes on Usage Classification of Chronic Pain (2nd Edition). Seattle, USA: IASP Press 1994.
5. Mundal I, GrÅwe R W, Bjørngaard J H, et al. Prevalence and long-term predictors of persistent chronic widespread pain in the general population in an 11-year prospective study: the HUNT study. BMC musculoskeletal disorders 2014; 15(1):1-12. doi: 10.1186/1471-2474-15-213
6. Treede R-D, Rief W, Barke A, et al. A classification of chronic pain for ICD-11. Pain 2015; 156(6):1003-07. doi: 10.1097/j.pain.0000000000000160
7. Kissin I. Long-term opioid treatment of chronic nonmalignant pain: unproven efficacy and neglected safety? 2013
8. Bradley L A. Pathophysiology of fibromyalgia. The American journal of medicine 2009; 122(12):S22-S30.
9. Xiao Y, Haynes W L, Michalek J E, et al. Elevated serum high-sensitivity C-reactive protein levels in fibromyalgia syndrome patients correlate with body mass index, interleukin-6, interleukin-8, erythrocyte sedimentation rate. Rheumatol Int 2013; 33(5):1259-64. doi: 10.1007/s00296-012-2538-6 [published Online First: 2012 Nov. 6]
10. Sturgeon J A, Darnall B D, Zwickey H L, et al. Proinflammatory cytokines and DHEA-S in women with fibromyalgia: impact of psychological distress and menopausal status. Journal of pain research 2014; 7:707-16. doi: [published Online First: 2014 Dec. 17]
11. Staud R. Evidence of involvement of central neural mechanisms in generating fibromyalgia pain. Curr Rheumatol Rep 2002; 4(4):299-305. [published Online First: 2002 Jul. 20]
12. Gur A, Karakoc M, Erdogan S, et al. Regional cerebral blood flow and cytokines in young females with fibromyalgia. Clin Exp Rheumatol 2002; 20(6):753-60. [published Online First: 2003 Jan. 2]
13. Gur A, Oktayoglu P. Status of immune mediators in fibromyalgia. Current pain and headache reports 2008; 12(3):175-81
14. Sjøgaard G, Søgaard K. Muscle injury in repetitive motion disorders. Clin Orthop 1998; 351:21-31
15. Brosschot J F, Gerin W, Thayer J F. The perseverative cognition hypothesis: A review of worry, prolonged stress-related physiological activation, and health. J Psychosom Res 2006; 60(2):113-24
16. Vlaeyen M S, Linton S J. Fear-avoidance and its consequences in chronic musculoskeletal pain: a state of the art. Pain 2000; 85(3):317-32. doi:
17. Tracy L M, Ioannou L, Baker K S, et al. Meta-analytic evidence for decreased heart rate variability in chronic pain implicating parasympathetic nervous system dysregulation. Pain 2016; 157(1):7-29
18. Cardiology TFotESo. Heart rate variability standards of measurement, physiological interpretation, and clinical use. Eur Heart J 1996; 17:354-81
19. Rainville P, Bechara A, Naqvi N, et al. Basic emotions are associated with distinct patterns of cardiorespiratory activity. Int J Psychophysiol 2006; 61(1):5-18
20. Martinez-Lavin M. Stress, the stress response system, and fibromyalgia. Arthritis Research and Therapy 2007; 9(4):216
21. Tan G, Fink B, Dao T K, et al. Associations among pain, PTSD, mTBI, and heart rate variability in veterans of Operation Enduring and Iraqi Freedom: a pilot study. Pain Medicine 2009; 10(7):1237-45
22. Furlan A D, Imamura M, Dryden T, et al. Massage for low back pain: an updated systematic review within the framework of the Cochrane Back Review Group. Spine 2009; 34(16):1669-84
23. Rosenzweig S, Greeson J M, Reibel D K, et al. Mindfulness-based stress reduction for chronic pain conditions: variation in treatment outcomes and role of home meditation practice. J Psychosom Res 2010; 68(1):29-36
24. Bawa F L, Mercer S W, Atherton R J, et al. Does mindfulness improve outcomes in patients with chronic pain? Systematic review and meta-analysis. Br J Gen Pract 2015; 65(635):e387-400. doi: 10.3399/bjgp15X685297 [published Online First: 2015 May 27]
25. Health. TNIoHNCfCaI. Terms Related to Complementary and Integrative Health https://nccih.nih.gov/health/providers/camterms.htm2016 [accessed May 9, 2016 2016
26. Kox M, van Eijk L T, Verhaak T, et al. Transvenous vagus nerve stimulation does not modulate the innate immune response during experimental human endotoxemia: a randomized controlled study. Arthritis research & therapy 2015; 17(1):1-9
27. Motley H L. The effects of slow deep breathing on the blood gas exchange in emphysema. Am Rev Respir Dis 1963; 88:484-92. doi: 10.1164/a rrd.1963.88.4.484 [published Online First: 1963 Oct. 1]
28. Mehling W E, Hamel K A, Acree M, et al. Randomized, controlled trial of breath therapy for patients with chronic low-back pain. Altern Ther Health Med 2005; 11(4):44-52. [published Online First: 2005 Aug. 2]
29. Jovanov E. On Spectral Analysis of Heart Rate Variability during Very Slow Yogic Breathing. Conference proceedings: Annual International Conference of the IEEE Engineering in Medicine and Biology Society IEEE Engineering in Medicine and Biology Society Annual Conference 2005; 3:2467-70. doi: [published Online First: 2007 Feb. 7]
30. Yasuma F, Hayano J. Respiratory sinus arrhythmia: why does the heartbeat synchronize with respiratory rhythm? Chest 2004; 125(2):683-90. [published Online First: 2004/02/11]
31. Kinabalu K. Immediate effect of ‘nadi-shodhana pranayama’ on some selected parameters of cardiovascular, pulmonary, and higher functions of brain. Thai journal of physiological sciences 2005; 18(2):10-16
32. Pramanik T, Pudasaini B, Prajapati R. Immediate effect of a slow pace breathing exercise Bhramari pranayama on blood pressure and heart rate. Nepal Med Coll J 2010; 12(3):154-57
33. Andersson U, Tracey K J. Reflex principles of immunological homeostasis. Annu Rev Immunol 2012; 30:313
34. De Couck M, Nijs J, Gidron Y. You may need a nerve to treat pain: the neurobiological rationale for vagal nerve activation in pain management. The Clinical journal of pain 2014; 30(12): 1099-105
35. Tracey K J. Reflex control of immunity. Nature Reviews Immunology 2009; 9(6):418-28
36. Tateishi Y, Oda S, Nakamura M, et al. Depressed heart rate variability is associated with high IL-6 blood level and decline in the blood pressure in septic patients. Shock 2007; 28(5):549-53
37. Tsutsumi T, Ide T, Yamato M, et al. Modulation of the myocardial redox state by vagal nerve stimulation after experimental myocardial infarction. Cardiovasc Res 2007
38. Pavithran P, Nandeesha H, Sathiyapriya V, et al. Short-term heart variability and oxidative stress in newly diagnosed essential hypertension. Clin Exp Hypertens 2008; 30(7):486-96
39. Tarapacki J, Thompson A, Kristal M B. Gastric vagotomy blocks opioid analgesia enhancement produced by placenta ingestion. Physiol Behav 1992; 52(1):179-82
40. Ben-Menachem E, Revesz D, Simon B J, et al. Surgically implanted and non-invasive vagus nerve stimulation: a review of efficacy, safety and tolerability. Eur J Neurol 2015; 22(9):1260-68. doi: 10.1111/ene.12629
41. Busch V, Zeman F, Heckel A, et al. The effect of transcutaneous vagus nerve stimulation on pain perception—an experimental study. Brain stimulation 2013; 6(2):202-09
42. Frokjaer J, Bergmann S, Brock C, et al. Modulation of vagal tone enhances gastroduodenal motility and reduces somatic pain sensitivity. Neurogastroenterol Motil 2016
43. Peuker E T, Filler T J. The nerve supply of the human auricle. Clin Anat 2002; 15(1):35-37
44. Frangos E, Ellrich J, Komisaruk B R. Non-invasive access to the vagus nerve central projections via electrical stimulation of the external ear: fMRI evidence in humans. Brain stimulation 2015; 8(3):624-36
45. Paccione C. Contemplative Cancer Care: A qualitative study of contemplative-based therapy for cancer patient wellbeing. Teachers College, Columbia University; Dept of Counseling and Clinical Psychology Columbia University 2043
46. Paccione C. Narrative Mindfulness. The Intima: A Journal of Narrative Medicine 2015 (E archive Fall 2015)

1. A device for alleviating or removing pain sensation and inflammation in an individual,
characterized in the device comprising a source for electricity (I) for creating pulses with a width range of 5-1000 μs, frequency of 1-50 000 Hz, voltage within an interval of 1.0-50,000 V, and a current within an interval of 0.01-10 mA to at least two electrodes (3.3′, 4) for creating a voltage between said electrodes, said electrodes being placed on a location on the body of the individual for stimulating the vagus nerve, the at least two electrodes (3.3′4) creating a voltage difference between said electrodes when connected to the power source (I),
the device further comprising sensors for monitoring and recording health measurements in order to optimize daily health, wherein the sensors are integrated in the device with electrodes that send data signals to a software application that collects and records health measurements.

2. A device according to claim 1, wherein the electrodes (3.3′, 4) are constructed to fit inside the ear (5) of the individual for affecting the vagus nerve.

3. A device according to claim 1 or 2, wherein the sensors may comprise at least one of biosensors, EEG (Electroencephalography) sensors/electrodes, Photoplethysmogram, Pulse oximeter, Electrocardiogram, infrared sensors, temperature sensors, and/or humidity sensors.

4. A device according to claim 3, wherein the collecting of data additionally is performed by using an accelerometer for measuring balance and lung capacity.

5. A device according to any of the previous claims, wherein the device is adapted to communicate with a smartphone or other smart devices to control, adjust, and monitor the device.

6. A device according to any of the previous claims, wherein the software application is equipped with artificial intelligence and algorithms that analyze this health data for users to track their own health.

7. A device according to any of the previous claims, wherein the biosensors are adapted to monitor body temperature, heart rate variability, respiratory rate, blood pressure, blood volume and blood oxygen saturation levels, capnography, brain waves, skin perspiration, focus and concentration, reaction time, and stress.

8. A device according to any of the previous claims, wherein the software application that collects and records health measurements from the device stores data on a cloud server that uses blockchain technology allowing users to share and privately own their health data.

9. A device according to any of the claims 1-8 for use in a method for treating or alleviating widespread chronic primary pain conditions.

10. A device according to claim 9, wherein said chronic primary pain and inflammatory conditions originate from stress, anxiety/depression, heart conditions, epilepsy, headaches/migraine, HRV (heart rate variations), inflammation, cognition disorders (e.g. dementia, Alzheimer's disease), stomach ailments (e.g. Crohn's disease, ulcer, chronic constipation, etc.), PTSD (post-traumatic stress disorder), idiopathic pain, weight gain/reduction, mood swings, post-treatment of drug withdrawal pain/symptoms and bacterial and/or viral diseases, particularly retrovirus such as AIDS and more particularly COVID19.

11. A device according to claim 9 for alleviating withdrawal symptoms in patients attempting to treat drug addiction including tobacco and alcohol addiction.

12. A device according to any of the claims 9-11 in a combination treatment program for alleviating or curing pain wherein the combination program includes activating the device while performing at least one breathing exercise.

13. A device according to claim 12, wherein the breathing exercise is selected from the group of mindfulness-based stress reduction (MBSR), contemplative pain treatment program (CPTP), contemplative breath-awareness practice, either relegated to awareness or compassion, Nadi Shodhana Pranayama or Bhramari pranayama.

14. A device according to claim 9 for treating animals afflicted with long term pain, afflicted with inflammation, afflicted with bacterial diseases and/or afflicted with viral diseases, for monitoring and optimizing the vital health of animals and research for the benefit of all domesticated and wild animals.
